(19) United States
(12) Patent Application Publication
(10) Pub. No.: US 2025/0132000 A1
(43) Pub. Date: Apr. 24, 2025

(54) SYSTEM AND METHOD TO ENHANCE THE CONTINUITY OF CARE FOR A PATIENT

(71) Applicant: Ocean Friends Inc., Irving, TX (US)

(72) Inventors: Animesh Kumar, Irving, TX (US); Manish Shukla, North Bend, WA (US)

(21) Appl. No.: 18/491,876

(22) Filed: Oct. 23, 2023

(51) Int. Cl. G16H 20/00; G16H 10/60

(52) U.S. Cl. CPC G16H 20/00; G16H 10/60

(57) ABSTRACT

A system and a method to enhance the continuity of care for a patient is disclosed. The method disclosed herein includes accessing the patient's data available in one or more formats, where the patient's data includes a pre-stored data, a real-time generated data or a combination thereof. The patient's data is then processed using machine learning techniques and language learning model by extracting relevant information from the patient's data. Further, the one or more patient-specific assignment recommendations are generated in correspondence to the processed patient's data, where the assignment recommendations are designed to address a condition management in the patient.

### TECHNICAL FIELD

The present disclosure relates to a system and method for enhancing continuity of care while managing a condition in a patient. In particular, the disclosure relates to automatically assigning tasks to the patient based upon therapy sessions conducted by an expert. The disclosure further allows a seamless communication between the user and the expert to enhance the outcome of the therapy session.

### BACKGROUND

Most often managing a health condition requires regular communication between a person and an expert to ensure proper support and monitoring. For example, a patient with chronic illnesses such as diabetes, hypertension, or cancer may require regular communication with healthcare professional to manage their condition, adjust treatments, and monitor progress. Similarly, a person aiming to lose weight may need to regularly consult a nutritionist or dietitian to achieve and maintain a healthy weight. In yet another example, a person with mental health issues, like depression, anxiety, or bipolar disorder, benefit from regular therapy or counseling sessions with mental health experts.

In many of the above health conditions, the caregivers fail to understand the manifestation of the condition in the person. Autism is one such neurodevelopmental condition that affects communication, social interaction, and behavior. People with autism often experience difficulties in interpreting social cues, making friends, and engaging in typical back-and-forth conversations. They may also exhibit repetitive behaviors, strict routines, and intense interest in specific subjects.

In the above scenarios, the person or his family may seek out support from an expert (e.g., a health professional, such as therapists or counselors, and others). This can help them manage the condition by ensuring that the person receives the best possible care.

One way of managing the abovementioned conditions is to provide effective therapy sessions to the person that involves comprehensive documentation and analysis of the person's progress and therapy plans. Traditionally, therapists create SOAP notes (Subjective, Objective, Assessment, and Plan), which help different experts such as therapists or clinicians communicate patient's progress in a standardized way. The notes also serve as proof of past therapies to the insurance companies.

Besides the treatment plans in the form of SOAP notes, home based exercises or between-session practice of the skills learned during the therapy is also necessary to make the treatment plan more effective. However, such home based exercises are often under recognized by the providers, the people undergoing therapies. This is mainly because the person, his parents and caregivers sometimes feel difficult to understand the actual method by which the home-based exercises is to be done by the person. In some cases, there is a lack of communication between the person, his family, and the expert, which leads to slow progress even after going through considerable number of therapy sessions.

Home based exercises (e.g., self-monitoring, behavior management, motor skills, speech therapy etc.) are assigned by the therapist or expert in-session and completed by the person before he comes for the next session. There are numerous benefits to the implementation of home-based exercises in the therapy process. Home exercises enables the generalization of skills and behaviors learned during therapy, facilitates treatment processes, provides continuity between sessions, and allows providers to better grasp patients learning.

Traditional methods of communication between therapists and parents are often limited to periodic in-person appointments, phone calls, or written instructions. These methods can lead to information gaps, misinterpretations, and a lack of real-time guidance. Existing technological solutions focus primarily on communication platforms or mobile applications that allow therapists to interact directly with patients. However, these solutions often exclude parents from the process, even though they play a pivotal role in ensuring therapy adherence and providing a conducive environment for home exercises.

Also, there are numerous barriers to the successful implementation of home exercises during treatment of the person have largely been suggested by experts in the field, rather than specifically measure, and have generally been classified as occurring on the provider-, patient-, task-, and environmental-level. Provider-level barriers can relate to the therapeutic relationship and the degree to which a collaborative approach is used, provider beliefs about home exercises and the patient's adherence, and providers ability to effectively design home assignments or exercises. Patient-level barriers can include patient avoidance and symptomatology, negative beliefs toward the task, not understanding the rationale or how to do the task, forgetting, and beliefs about their ability to complete home based exercises or tasks. Relatedly, core beliefs central to the patients' psychopathology can be activated during home exercises-thereby triggering withdrawal and avoidance patterns. Task-level barriers include poor match between tasks and therapy goals, tasks that are perceived as vague or unclear, tasks that are perceived as too difficult or demanding in terms of time or effort, tasks being viewed as boring, and general aversiveness of the idea of completing home exercises. Environmental factors have been noted to include practical obstacles, lack of family/caregiver support, dysfunctional home environments, lack of time due to busy schedules, and lack of reward or reinforcement.

Therefore, there is a need for a system and a method for seamless communication between the person, his parent and caregiver and the expert in order to enhance the continuity of care for a patient. Furthermore, the system should be easy to use by the person or any other user appointed to interact with the expert.

### SUMMARY

The present invention discloses a system and method to enhance the continuity of care for a patient. In particular, the disclosure relates to generating one or more home assignment recommendations for the patient which is communicated to the parent and caregiver of the patient and provides a seamless communication between the parent and the expert.

In one aspect of the present invention, a method to enhance the continuity of care for a patient is disclosed. The method disclosed herein includes accessing the patient's data available in one or more formats, where the patient's data includes a pre-stored data, a real-time generated data or a combination thereof. The patient's data is then processed using machine learning techniques and language learning model by extracting relevant information from the patient's data. Further, the one or more patient-specific assignment recommendations are generated automatically in correspondence with the processed patient's data, where the one or more assignment recommendations are designed based on a correlation between the pre-stored data and real-time generated data in order to address a condition management in the patient. The one or more input is received from the expert on one or more assignment recommendations in order to generate one or more approved assignment recommendations. The approved one or more assignment recommendations is communicated to one or more remote users including, parents and caregivers of the patient, where the parents and caregivers may access and review the one or more approved assignment recommendations.

In another aspect of the present invention, a system to enhance the continuity of care for a patient is disclosed. The system includes a server and a processing device that is operatively coupled to the server. The server includes a memory to store the medical data of the patient, while the processing device executes the instructions to: access the patient's data available in one or more formats, where the patient's data includes a pre-stored data, a real-time generated data or a combination thereof. The patient's data is then processed using machine learning techniques and language learning model based on extraction of relevant information from the patient's data. The one or more patient specific assignment recommendations are generated automatically in correspondence with the processed patient's data, where the one or more assignment recommendations are designed based on a correlation between the pre-stored data and real-time generated data in order to address a condition management in the patient. Further, the one or more input is received from the expert on one or more assignment recommendations in order to generate one or more approved assignment recommendations The approved one or more assignment recommendations is communicated to one or more remote users which may include parents and caregivers of the patient, where the parents and caregivers can access and review the one or more approved assignment recommendations.

In an embodiment, the one or more approved assignment recommendations may be communicated to the remote users in one or more of the following formats-text, audio, video, image, gaming task or a combination thereof.

In yet another embodiment, the patient's data may be in one or more of the following formats text, audio, video, image, recording of the session or a combination thereof.

Advantageously, the user interface may include a graphical user interface, a voice user interface, a gesture user interface, or any suitable user interface. Also, the user may provide a text input, a voice command, or a gesture as an input.

### BRIEF DESCRIPTION OF THE ACCOMPANYING DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

FIG. 1 illustrates a system for enhancing the continuity of care for a patient, in accordance with one implementation of the present disclosure.

FIG. 2 depicts an exemplary implementation of the data model for enhancing continuity of care for patient, in accordance with one implementation of the presents disclosure.

FIG. 3 depicts an exemplary implementation disclosing the working of the user interface and data model in generating one or more assignment recommendations, in accordance with one implementation of the present disclosure.

FIG. 4 illustrates a method for enhancing the continuity of care for a patient, in accordance with one implementation of the present disclosure.

FIG. 5 illustrates an exemplary method for enhancing the continuity of care for a patient by communicating different types of assignment recommendations to the parent of the patient, in accordance with one implementation of the present disclosure.

FIG. 6a illustrates an exemplary method for generating an assignment recommendation to a patient undergoing therapy, in accordance with one implementation of the present disclosure.

FIG. 6b illustrates an exemplary method which discloses an interaction between the parent and an expert when the patient faces problems while completing the assignment, in accordance with one implementation of the present disclosure.

FIG. 6c illustrates an exemplary method which discloses an interaction between the parent and an expert when the patient does not complete the entire assignment, in accordance with one implementation of the present disclosure.

FIG. 6d illustrates an exemplary method which discloses an interaction between the parent and an expert when the patient does not complete the entire assignment and the notification is shared to the parent before the next session, in accordance with one implementation of the present disclosure.

FIG. 7 illustrates an exemplary implementation which discloses how a notification of new assignment recommendations is communicated on parents and caregiver device, in accordance with one implementation of the present disclosure.

### DETAILED DESCRIPTION

Embodiments, of the present disclosure, will now be described with reference to the accompanying drawing.

In the following description, certain specific details are set forth in order to provide a thorough understanding of various disclosed embodiments. However, one skilled in the relevant art will recognize that embodiments may be practiced without one or more of these specific details, or with other methods, components, materials, etc.

Unless the context indicates otherwise, throughout the specification and claims which follow, the word “comprises” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context clearly dictates otherwise.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Assignment activities (e.g., self-monitoring, behavior management, motor skills, speech therapy etc.) are assigned by providers in-session and completed by a patient in-between sessions with the goal of “practicing” therapeutic skills in the environment where it will be most needed. There are numerous benefits to the implementation of assignment in the treatment process. Assignment enables the generalization of skills and behaviors learned during therapy, facilitates treatment processes, provides continuity between sessions, allows providers to better grasp patients learning, and strengthens that learning, leading to improved maintenance of treatment gains.

While the description presented herein makes a specific reference to Autism it is to be appreciated that aspects of the present disclosure are also equally applicable to other conditions such as Dementia, Alzheimer's, Parkinson, or any other condition and may deal with any kind of patient other than child.

The system and method disclosed herein includes a method to enhance the continuity of care for a patient. The system and method disclosed herein includes accessing the patient's data available in one or more formats, where the patient's data includes pre-stored data, real-time generated data or a combination thereof. The pre-stored data may include one or more medical session reports or progress reports of the patient including medical session notes (e.g., SOAP notes) of the previous sessions indicating assessment of the condition and progress made by the patient in previous sessions. The real-time generated data includes the progress notes including SOAP notes of the ongoing session, medical data of one or more patients with similar condition or a combination thereof. The patient's data is a combination of medical session report, a progress report, patient's health record or a combination thereof. The medical session report includes one or more previous or ongoing session reports, the progress report includes a combination of one or more medical session reports to define progress made by the patient over a period, and patient's health record includes one or more additional health related details of the patient. Further, the medical session report and progress report includes one or more SOAP notes which basically includes the details related to subjective information, objective observations, assessment findings, plans for future treatment of the patient and optionally includes one or more assignment recommendations. The patient's data is then processed using machine learning techniques and language learning model by extracting relevant information from the patient's data. Further, the one or more patient-specific assignment recommendations are generated automatically in correspondence with the processed patient's data, where the one or more assignment recommendations are designed based on a correlation between the pre-stored data and the real-time generated data in order to address a condition management in the patient. The one or more input is then received from the expert in order to generate one or more approved assignment recommendations. The approved one or more assignment recommendations is communicated to one or more remote users including, parents and caregivers of the patient, where the parents and caregivers may access and review the one or more assignment recommendations. The communication method allows the parents and caregivers to communicate with the expert while the patient works on the one or more approved assignment recommendations, thereby enhancing the continuity of care by providing patient's engagement and condition management.

It should be noted that the description here is not only limited to parents and caregivers of the patient, but it may also include family members, extended family members, teachers of the patient, other therapists, doctors, coaches of the patient (if the patient is undergoing any other treatment with some other doctor, therapist) and so on. Also, the term ‘expert’ disclosed herein may include any person who has experience in that specific field and may include other persons like doctors, therapists, clinicians, nurses, physicians and so on.

Further the one or more assignment recommendations generated herein is communicated to the parents and caregivers of the patient in various formats which may include text-based assignment where one or more approved assignment recommendations is provided to the patient in the form of text, for example, “Andrew will have to spell /er/ 5 times” and so on. The other format of one or more approved assignment recommendations include audio, video, games, web links to the music albums, web links to the games, sensory toys and their guides, listening programs like bone conduction headphone and so on. These details are shared on the parents and caregiver's device and also on the dashboard where the patient's medical record exist. All the one or more approved assignment recommendations are generated automatically using data model. The various types and formats of assignment are discussed in detail below.

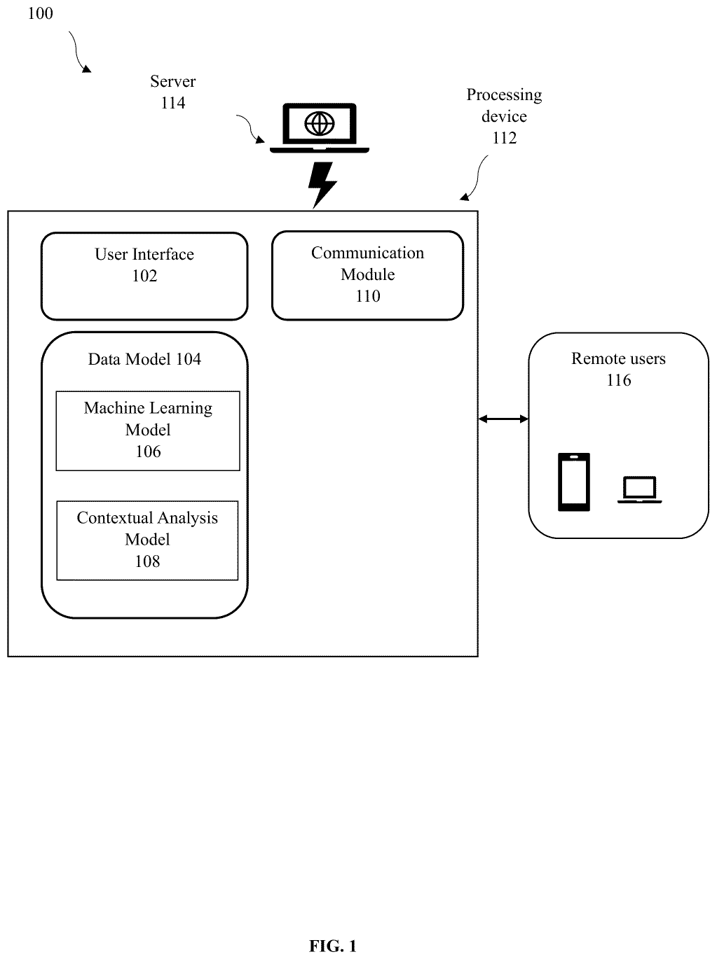

FIG. 1 illustrates a computer implemented system 100 used for enhancing the continuity of care for a patient, in accordance with one implementation of the present disclosure. While the system 100 discussed here refers to enhancing the continuity of care for a patient, it should be noted that the system 100 may also be used for additional applications where a user interacts with an expert for management of conditions related to, but not limited to, health and wellness, education, technical support and any other consultation.

The system 100 includes one or more components that work in tandem to provide a seamless user experience. In the shown embodiment, the system 100 includes a processing device 112 that is communicatively coupled to a server 114. As shown, the processing device 112 includes a user interface 102, a data model 104 and a communication module 110. The data model 104 further includes a machine learning model 106 and a contextual analysis model 108 for automatically generating the one or more assignment recommendations for the patient that is to be completed by the patient in-between the two sessions. The server 114 includes a memory (not shown) that stores details of the patient's past medical record, SOAP notes of the previous and the ongoing sessions, one or more assignment recommendations allotted to the patient in the past and the ongoing sessions, and so on. The communication module 110 is configured to communicate the automatically generated one or more assignment recommendations to the one or more remote user 116. The system 100 further includes one or more remote users 116, where the remote users 116 may be the patient, parents and caregivers of the patient, guardian of the patient, and so on. The one or more remote users 116 interacts with the system 100 using a computing device including, but not limited to, a mobile device, computer, tablet, laptop and so on.

The processing device 112 serves as a primary interface between the expert and the one or more remote user 116. The user interface 102, data model 104 and communication model 110 are operatively coupled to each other. The user interface 102 is designed to be intuitive and easy to use, enabling the expert and one or more remote users 116 to access the patient's data, SOAP notes of the previous and ongoing sessions, one or more assignment recommendations and so on without any difficulty. The user interface 102 allows an expert to access the patient's data in one or more formats which may include text, image, audio, video or a combination thereof.

The system 100 disclosed herein allows an expert to gain access through the commonly used user interfaces 102 which may include a graphical user interface, a voice user interface, a video user interface, a gesture user interface, or any suitable user interface. In an implementation, the expert may access an image input or video input of the patient to the user interface 102. Based on the patient's data accessed by the expert the data model 104 will process the input data and automatically generate one or more approved assignment recommendations for the patient that is to be performed in-between the sessions in order to maintain the continuity of care.

The system 100 includes a user interface 102 and a data model 104 that are operatively coupled to each other using which the expert can access the patient's data and automatically generate one or more assignment recommendations. The expert may simply open a platform (may also be referred as ‘Ocean Friend's Dashboard’) and access the patient's data which mainly includes pre-stored data, real-time data or a combination thereof. The pre-stored data may include one or more session notes, medical session reports, and/or progress reports of the patient. In one example, the pre-stored data may include SOAP notes of the previous sessions indicating assessment of the condition and progress made by the patient in previous sessions. The real-time generated data includes a progress report of the patient which includes SOAP notes of the ongoing session, medical data of one or more patients with similar condition or a combination thereof. The patient's data is a combination of a session notes, medical session report, progress report, or patient's medical health record. The medical session report includes one or more previous or ongoing session reports, the progress report includes combination of one or more medical session reports that define progress made by the patient over a period of time, and patient's health record includes one or more additional health related details of the patient. Further, the medical session report and progress report includes one or more SOAP notes, which basically includes details related to subjective information, objective observations, assessment findings, plans for future treatment of the patient and optionally includes one or more assignment recommendations.

The system uses machine learning model 106 to automatically generate the one or more assignment recommendations based on the extraction of relevant data from the patient's previously generated SOAP notes, SOAP notes of ongoing session, one or more assignment recommendations in the previous sessions, medical data or one or more assignment exercises of one or more other patients dealing with a similar condition. The generated one or more assignment recommendations is then reviewed and approved by the expert, where the expert provides one or more inputs in order to generate one or more approved assignment recommendations.

The system 100 allows contextual analysis of the patient's data using fine-tuned large language models and machine learning techniques. Such analysis allows extraction and categorization of the patient's data accessed by the expert into its suitable category which are pre-defined under Subjective, Objective, Assessment, Planning and Assignment Recommendations. The contextual analysis module 108 make use of machine learning module 106 in comparing the patient's real-time data with the pre-stored data stored in the repository in order to find which data falls under which category. Further details pertaining to the categorization of the patient's data to generate one or more assignment recommendations will be discussed in subsequent section.

The processing device 112 of the system 100 can be any device that is equipped with the necessary components which allows an expert to access the patient's data using a user interface 102, generate one or more assignment recommendations using the machine learning model 106 and contextual analysis model 108 which are a part of the data model 104 of the system 100. Further, the one or more generated assignment recommendations are reviewed by the expert and changes are made to them, if needed by providing one or more input from the expert. Finally, the approved one or more assignment recommendations is communicated to one or more remote users 116 using a communication module 110. The one or more remote users 116 may include parents, caregivers, or a guardian of the patient. Exemplary device may be—smartphone, tablet, personal computer, and so on. With its advanced technology and user-friendly user interface 102, the processing device 112 can provide an ideal platform for the expert and one or more remote users 116 to interact with each other.

The system 100 also allows one or more remote users 116 to directly access the platform using user interface 102 and using this the one or more remote users 116 may get to know the progress of treatment, one or more assignment recommendations generated for the child by the expert and other health condition related details of the patient. The one or more remote users 116 may also interact with the expert using the user interface 102 in case of any queries related to a health condition of patient or any queries related to one or more assignment recommendations.

The data model 104 is operatively coupled with the user interface 102 and the communication module 110. It further consists of machine learning module 106 and contextual analysis module 108. The data model 104 refers to the part of the system 100 which is responsible for processing the patient's data accessed by the expert. The machine learning module 106 present in the data model 104 of the system 100 is configured to process the patient's data accessed by the expert using machine learning techniques and language learning model to extract the relevant information from the patient's data. The machine learning model 106 generates the one or more assignment recommendations based on the SOAP notes of the ongoing and previous sessions of the patient, medical data which may include SOAP notes of one or more other patients dealing with similar condition. The patient's data mainly include pre-stored data and real-time generated data. The pre-stored data may include one or more session notes, medical session reports, or progress reports of the patient, which may include SOAP notes of the previous sessions indicating assessment of the condition and progress made by the patient in previous sessions. The real-time generated data includes SOAP notes of the patient from the ongoing session, medical data of one or more patient dealing with similar condition or a combination thereof. The patient's data is a combination of session notes, a medical session report, a progress report, and the patient's medical health record. The medical session report includes one or more previous or ongoing session reports, the progress report includes a combination of one or more medical session reports that define progress made by the patient over a period, and patient's health record includes one or more additional health related details of the patient. Further, the medical session report and progress report may include one or more SOAP notes, which basically includes details related to subjective information, objective observations, assessment findings, plans for future treatment of the patient and optionally includes one or more assignment recommendations. The machine learning module 106 compares the real-time generated data i.e., the progress report of the ongoing session with the pre-stored data and automatically generates one or more assignment recommendations. The one or more assignment recommendations are then reviewed by the expert and may be edited, if needed, by providing one or more input from the expert. This will help in generating the one or more approved assignment recommendations.

The data model 104 allows an expert to access the patient's data from the user interface 102 in one or more formats. The patient's data may be available to the expert in various formats like text, image, audio, video, recording from the session or a combination thereof. The data model 104 is configured to identify the intent behind the patient's data. To that end, the data model 104 further includes a contextual analysis module 108 for analyzing the patient's data. The contextual analysis module 108 is configured to extract relevant information under different categories, which will help machine learning module 106 to automatically generate one or more assignment recommendations.

The data model 104 allows the expert to access the patient's data in one or more formats using the user interface 102. The patient's data may be in the form of text, video, image, audio, recording from the session or a combination thereof. The patient's data mainly include pre-stored data and real-time generated data. The pre-stored data includes one or more medical session reports or progress reports of the patient including SOAP notes of the previous sessions indicating assessment of the condition and progress made by the patient in previous sessions. The real-time generated data include SOAP notes of the patient from the ongoing session, medical data of one or more other patients dealing with similar situation. The patient's data is a combination of a medical session report, a progress report, patient's medical health record or a combination thereof. The medical session report includes one or more previous or ongoing session reports, the progress report includes one or more medical session report clubbed to define progress made by the patient over a period, and patient's health record includes one or more additional health related details of the patient. Further, the medical session report and progress report includes one or more SOAP notes, which basically includes details related to subjective information, objective observations, assessment findings, plans for future treatment of the patient and optionally includes one or more assignment recommendations. This may involve use of machine learning algorithms or other forms of artificial intelligence to analyze the patient's data, access relevant databases and knowledge sources, and generate an appropriate response. Here, the database may include a repository of information obtained from previous sessions of the patient or other patients. This may include the information provided by the patient about the condition or the recommendations made by the clinician about the prognosis and therapy of various patients. Typically, the database is the repository of patients records that include all the information about patients. This information may be generated by clinicians or health care professionals based upon direct or indirect interaction with the patients or individuals having knowledge of patient's conditions. Some exemplary databases may include clinical data repository (CDR) or clinical data warehouse (CDW), and electronic health records (EHRs).

The data model 104 is a crucial component of the system 100, as it determines the behavior of the user interface 102 and the quality of the automatically generated one or more assignment recommendations that it provides to the one or more remote users 116. The data model 104 should be designed to provide accurate, relevant, and helpful suggestions to the expert in real-time. The data model 104 disclosed herein is scalable, efficient, and robust, so that it can handle a large number of inputs and provide quick and accurate responses to the expert as well as one or more remote users 116. In an exemplary embodiment, three assignments are allotted to the child to be completed and submitted before the next session. Suppose the child is showing a negative impact while performing the 2^{nd} task, so in that case the parent may directly connect to the expert via the user interface 102. The parent may reach out stating “Child is facing problem in completing 2^{nd} task”. The data model 104 will analyze the input and will provide a new assignment recommendation to the parent stating “Stop child from doing 2^{nd} task and let her stay calm for some time”. Further, the data model 104 will allot some new assignment to the child which would be different from the one provided earlier, since the child was not showing a positive impact with the given assignment.

Further, the communication module 110 enables the processing device 112 to communicate with one or more remote users 116 which may include parents, caregivers, guardians of the patient, and so on. The communication module 110 is operatively coupled to the user interface 102. The communication module 110 ensures that the processing device 112 can receive and transmit data from and to a device of the remote user. This allows remote users 116 to keep a track of patient's therapy, assignment recommendations, patient's medical data, and so on. The one more remote users 116 may also interact with the expert in case of any query related to patient's health. In an example, a child is provided three task to complete in-between the therapy sessions. The child has completed two tasks and becomes aggressive while doing the remaining task, then the parent of the child may interact with the expert via the user interface 102 and the data model 104 analyze and revert based on the inputs provided by the parent, past medical record of the child, other patients dealing with similar condition, or a combination thereof.

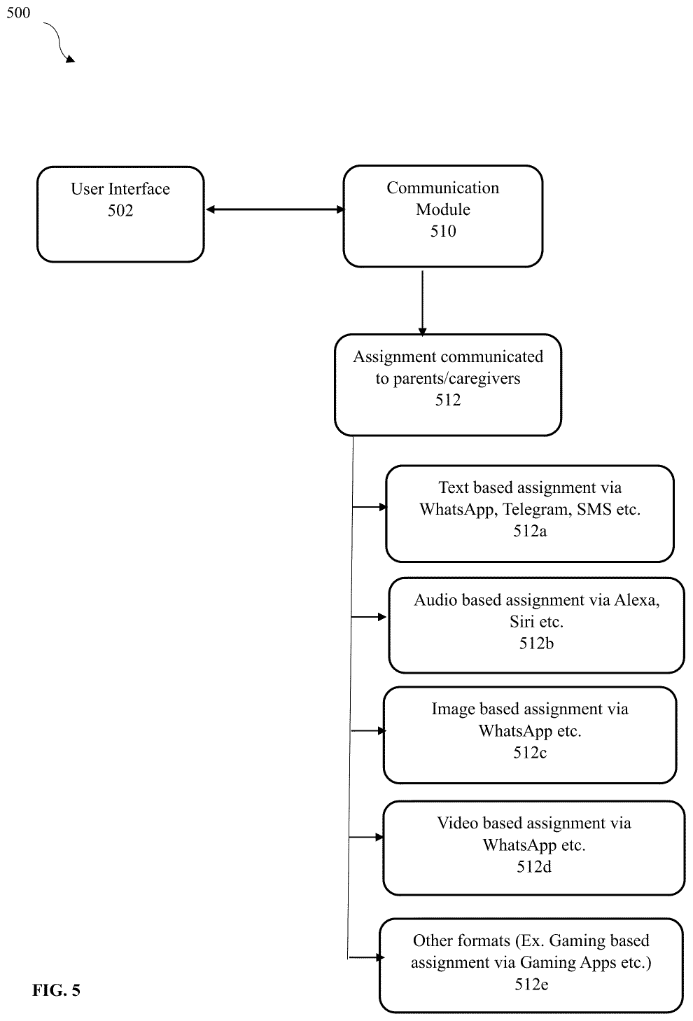

The automatically generated assignment recommendations may be communicated to one or more remote users 116 in plurality of formats after getting reviewed and approved by the expert. This may include image, text, audio, video, others and a combination thereof. The same is explained in detail in FIG. 5.

To that end, the communication module 110 should be designed to be flexible and adaptable, allowing the user interface 102 to communicate the one or more approved assignment recommendations in the format that is most convenient for the one or more remote user to understand, and in a way that is easily accessible and understandable. The communication module 110 should also be designed to be secure, ensuring that the patient's information and data remain confidential and protected.

In this scenario, the processing device 112 communicates with the server 114 via a communication network. The server 114 acts as a backbone of the system 100, which stores the patient's data including a combination of medical session report, progress report, patient's medical health record or a combination thereof. The medical session report includes one or more previous or ongoing session reports, the progress report includes one or more medical session report clubbed to define progress made by the patient over a considered period, and patient's health record including additional health related details of the patient. Further, the medical session report and progress report includes one or more SOAP notes, which basically includes details related to subjective information, objective observations, assessment findings, plans for future treatment of the patient and optionally includes one or more assignment recommendations. Also, in this scenario, a user profile is created whenever the physician/therapist creates a new and unique profile of a new patient. The user profile includes information related to the patient's health conditions, device related information, physician/therapist inputs, one or more assignment recommendations details and generated SOAP notes. It may also be noted that in such scenario, the all such information is stored in the memory for any future use.

The system 100 for enhancing the continuity of care for a patient is designed to provide individuals with an effective way to maintain the progress of their condition and provides a seamless communication between the expert and the one or more remote users 116. The natural language processing technology used by the system 100 ensures that the one or more assignment recommendations allotted to the patient is clearly understood by the parent and caregiver of the patient. To that end, if the parents or caregiver need any support or have any queries related to the recommended assignments, they may interact with the expert directly via the disclosed platform.

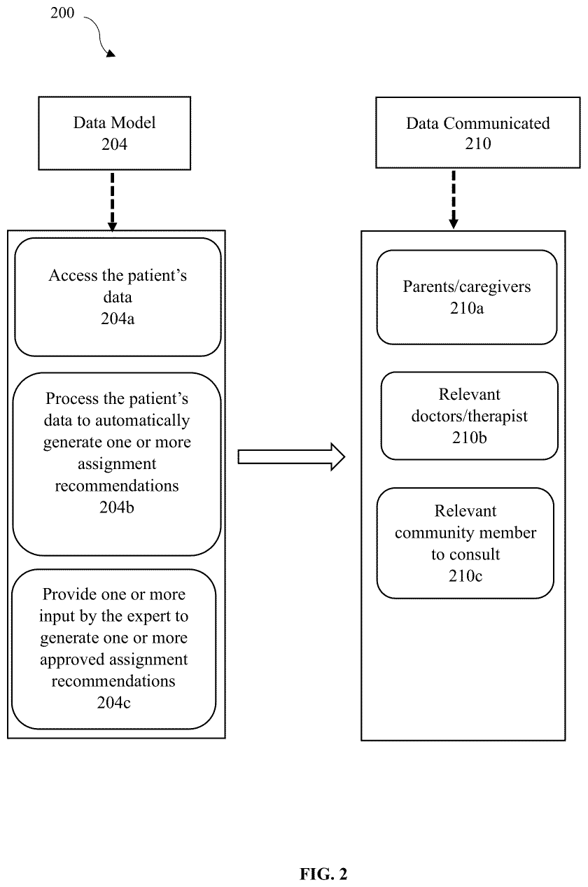

FIG. 2 depicts an exemplary implementation 200 of data model 204 for enhancing continuity of care in patients, in accordance with one implementation of the present disclosure.

As discussed previously, the expert may access patient's data 204a that is available to the expert in various formats via user interface (not shown in FIG. 2). The patient's data is provided in one or more of the following forms-text input, image input, audio input, video input, recording from the session, or a combination. The patient's data is then provided to the data model 204 for further processing. Data model 204 then the patient's data by extracting relevant information from patient's data. The patient's data may include pre-stored data or real-time generated data, where pre-stored data may include one or more medical session reports or progress reports of the patient including session notes (e.g., SOAP notes, therapy notes, etc.) of the previous sessions indicating assessment of the condition and progress made by the patient in previous sessions 204. And real-time data includes SOAP notes of the ongoing session, medical data of one or more patients dealing with similar situation, or a combination thereof. The patient's data is a combination of a medical session report, a progress report, patient's medical health record or a combination thereof. The medical session report includes one or more previous or ongoing session reports, the progress report includes one or more medical session report clubbed to define progress made by the patient over a period, and patient's health record includes one or more additional health related details of the patient. Further, the medical session report and progress report includes one or more SOAP notes, which includes details related to subjective information, objective observations, assessment findings, plans for future treatment of the patient and optionally includes one or more assignment recommendations.

The machine learning module present in the data model 204 analyzes and compares the real-time data of the patient with the pre-stored data and automatically generates one or more assignment recommendations for the patient 204b. The one or more generated assignment recommendations are then reviewed by the expert 204c, which may include editing or modifying the one or more assignment, if needed. The expert may make changes in the one or more assignment recommendations automatically generated by the data model 204, in case if any changes are needed by providing one or more input. The one or more approved assignment recommendation is generated after receiving the one or more input from the expert.

The one or more approved assignment recommendations are then communicated 210 to one or more remote users which may include relevant parents, caregivers 210a, relevant doctors, therapist 210b and/or relevant community member for consultation 210c.

Further, the data model 204 is configured to handle different formats of data, including text, audio, image, video and so on. Also, the data model 204 can handle unstructured data. The data model 204 can be a machine learning model and may be configured to process and analyze fed data using techniques such as language learning models and NLP (natural language processing). The data model 204 may also define the input and output format of the data and how it is processed by the machine learning algorithm.

Further, the data model 204 can learn and improve over time, as it gets exposed to more inputs by physician/therapist, response, and other related data. The data model 204 is trained using large amounts of data and sophisticated machine learning techniques, which enables the data model 204 to learn patterns and relationships within the data. Further, the data model 204 gets updated each time the expert provides a new input or a corresponding SOAP note is generated which further helps in generating an updated set of one or more assignment recommendations for the patient which are then approved by the expert to generate one or more approved assignment recommendations for communication to one or more remote users.

The data model 204 disclosed is configured to learn and adapt to new inputs and feedback from expert. In an example, if the patient consistently provides positive response for certain assignment recommendations, the data model 204 will analyze the set of tasks and provide similar tasks to the patient, as it is helping in patient's growth and treatment. Similarly, if the patient consistently provides negative response for certain assignment recommendations, the data model 204 will analyze the set of tasks and will not provide similar tasks to the patient, as it is hindering in patient's growth and treatment.

Exemplary data models 204 uses Generative AI model which may include GPT-3 (Generative Pre-trained Transformer 3), GPT-3.5, BERT (Bidirectional Encoder Representations from Transformers), ROBERTa (Robustly Optimized BERT Approach), ALBERT (A Lite BERT), T5 (Text-to-Text Transfer Transformer), or any other suitable models known to those skilled in the art.

In summary, data model 204 can handle different types of input data, including images and sound, along with text, as long as the structure and relationships between the data is properly defined in the data model 204.

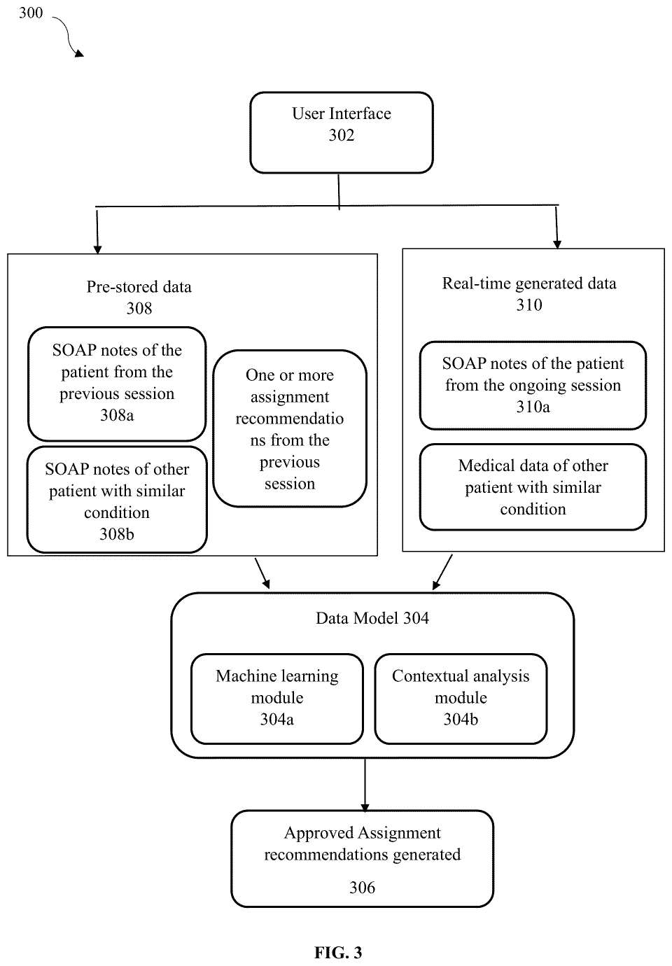

FIG. 3 depicts an example implementation disclosing the working of the user interface and data model in generating one or more assignment recommendations, in accordance with one implementation of the present disclosure.

It discloses the working of user interface 302 and data model 304. The user interface 302 and the data model 304 are operatively connected to each other. The patient's data can be accessed by the expert in one or more formats. The patient's data is available in formats like image, text, audio, video, recording of the session or a combination thereof. The patient's data available in the user interface 302 includes pre-stored data 308 and real-time generated data 310. The pre-stored data 308 includes SOAP notes of the patient from the previous sessions 308a, SOAP notes of one or more other patients dealing with similar condition 308b, one or more assignment recommendations for the patient from the previous sessions 308c or a combination of these. The real-time generated data 310 includes SOAP notes of the patient from the ongoing session 310a, SOAP notes or medical data of one or more other patient dealing with similar condition 310b or a combination thereof. The patient's data which can be accessed by the expert via user interface 302 is transferred to the data model 304 for further processing.

The data model 304 is operatively coupled to the user interface 302. The data model 304 further includes a machine learning module 304a and a contextual analysis module 304b which uses the patient's data available in one or more formats in the user interface 302 and is further configured to automatically generate one or more assignment recommendations 306. The machine learning module 304a analyzes and compares the real-time generated data 310 with pre-stored data 308 and extracts the relevant information from the patient's data. Further, the machine learning module 304a makes use of contextual analysis module 304b to categorize the extracted patient's data into its specific category. As a result, the data model 304 by making use of machine learning module 304a and contextual analysis module 304b automatically generates one or more assignment recommendations 306 which is communicated to one or more remote users. Before communicating the one or more assignment recommendations they are reviewed by the expert. The expert may edit and/or modify the one or more assignment recommendations, if needed by providing one or more input. This would be clearer by the following example, suppose the data model 304 has ‘generated 5 tasks as assignment’ to be completed before the next session. But the expert feels that the amount of assignment allotted to the child is too much, as the parent of the child has reported to expert via a user interface 302 that ‘the child has undergone a surgery’. So, the expert may reduce the amount of assignment or may allot some simple tasks to the child.

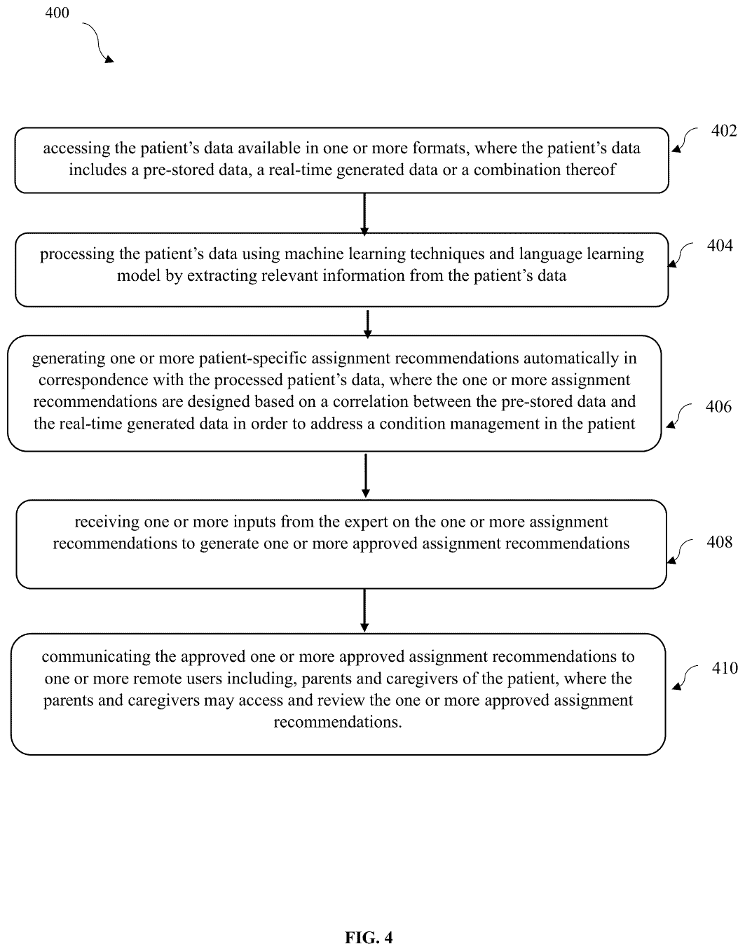

FIG. 4 illustrates a method 400 for enhancing the continuity of care in a patient, in accordance with one implementation of the present disclosure.

As shown, the method 400 for enhancing the continuity of care for a patient include following steps:

At 402, the expert accesses the patient's data in one or more formats, where the patient's data includes a pre-stored data, a real-time generated data or a combination thereof. The patient's data may be available in the form of a progress report which includes a SOAP note which further includes details related to subjective information, objective observations, assessment findings, plans for future treatment of the patient. The pre-stored data may include medical records of the patient including one or more session reports, session notes, progress reports, conversation of the patents and/or patients with the expert, previous assignment recommendations, and so on. The real-time generated data may include the session notes or medical session report of the ongoing session, or medical data of one or more other patients dealing with similar condition or having similar manifestation of the condition, either alone or in combination. The patient's data can be a combination of medical session reports, progress reports, patient's medical health record. The medical session report includes one or more previous or ongoing session reports, the progress report includes combination of one or more medical session reports that defines progress made by the patient over a period of time, and patient's health record includes one or more additional health related details of the patient. Further, the medical session report and progress report can include one or more SOAP notes, which basically includes details related to subjective information, objective observations, assessment findings, plans for future treatment of the patient and optionally includes one or more assignment recommendations. The patient's data may be in present in one or more formats which may include text, audio, video, image or a combination thereof.

Further, at 404 the patient's data is processed using machine learning techniques and language learning model by extracting relevant information from the patient's data. The patient's data is processed using the data model which further includes machine learning model and contextual analysis model. Further, correlating the patient's data with the pre-stored data and real-time generated data may include use of generative AI models (such as large language models or LLMs like ChatGPT, Bard, etc.) and/or use of one or more relevant machine learning modules. For instance, the generative AI may use the pre-stored data to generate one or more assignment recommendations. To that ends, the generative AI model may correlate the received patient's data and correlate the same with the data stored in the database to generate a more comprehensive and accurate one or more assignment recommendations. For example, the generative AI model may consider the prognosis of the past medical session reports and the one or more approved assignment recommendations allotted to the patient to generate one or more assignment recommendations of ongoing session. It should be noted that the generative AI model may include one or more fine-tuned large language models including ChatGPT, Bard, and others. In another instance, the correlation of patient's data with the pre-stored data may be done using a machine learning module. Here, the machine learning module may identify the patterns from the patient's data stored in the database to generate relevant one or more assignment recommendations. The module may also make assignment recommendation based on adaptation of assignment recommendations made to other patients in the past, where the other patients had similar manifestation of the condition as of the patient under consideration.

At 406 one or more patient-specific assignment recommendations are automatically generate in correspondence with the processed patient's data. Here, the one or more assignment recommendations are generated based on a correlation between the pre-stored data and real-time generated data in order to manage a condition in the patient. The one or more assignment recommendations are generated automatically using data model which further includes machine learning model and contextual analysis model. The automatically generated one or more assignment recommendations is generated based on the SOAP notes of the ongoing and previous sessions, medical data of one or more patients with similar condition and are tailored according to the patient's requirement.

At 408, one or more inputs from the expert are received on the generated one or more assignment recommendations, this leads to generation of one or more approved assignment recommendation.

Furthermore, at 410, the approved one or more assignment recommendations are communicated to one or more remote users including, parents and caregivers of the patient, where the parents and caregivers may access and review the one or more assignment recommendations. The one or more approved assignment recommendations communicated to the one or more remote user may be in one or more format which may include text-based assignment, image-based assignment, video-based assignment, audio-based assignment, others like gaming task or a combination thereof.

The communication method 400 allows the parents and caregivers to communicate with the expert while the patient works on the one or more approved assignment recommendations, thereby enhancing the continuity of care by providing the patient's engagement and condition management.

The one or more remote user may initiate an update to the expert on completing the one or more approved assignment recommendations, thereby facilitating a seamless communication between the one or more remote user and the expert. The communication method 400 allows one or more remotes users to establish a user-query interface to enable users to pose questions or seek clarification from the expert regarding the one or more approved assignment recommendations and/or one or more health condition of the patient. The interface allows the user to interact with the expert to seek any clarifications on the assignment recommendations. The expert may be a physician, therapist, clinician, doctor and/or any other person having experience in providing recommendations and/or treatment related to the condition of the patient. The expert may address one or more remotes user queries, provide guidance and maintain an interactive therapeutic consultation with the user in order to optimize treatment continuity and enhance the interaction between the user and expert.

FIG. 5 illustrates an exemplary method for enhancing the continuity of care for a patient by communicating different types of assignment recommendations to the parent of the patient, in accordance with one implementation of the present disclosure.

The example focusses on different formats of one or more approved assignment recommendations allotted to the patient. As shown, communication module 510 and user interface 502 are operatively coupled to each other. The communication module 510 communicates automatically generated one or more approved assignment recommendations to one or more remote users via the interface 502, in one or more formats.

The expert may access the patient's data via the user interface 502 based on which the data model (not shown in FIG. 5) automatically generates one or more assignment recommendations. The automatically generated one or more assignment recommendations is then edited by the expert to generate approved assignment recommendations, which is then communicated to one or more remotes users. The one or more remote users may include parents, caregivers, guardian of the child and so on.

The communication module 510 communicates the automatically generated one or more approved assignment recommendations to the one or more remotes users, parents and caregivers 512 in this case. The one or more approved assignment recommendations may be communicated to parents and caregivers 512 in one or more formats. For example, the assignment recommendations may be in the form of text-based assignment 512a, audio-based assignment 512b, image-based assignment 512c, video-based assignment 512d, or other formats 512e such as game links, or a combination thereof. The text-based assignment 512a may be communicated to parents and caregivers 512 either on the platform where the profile of the patient exists or directly to the device of the parents and caregiver 512 using messaging Apps like WhatsApp, Telegram, SMS and so on. The audio-based assignment 512b may be communicated to parents and caregivers 512 either on the platform where the profile of the patient exists or directly to the device of the parents and caregiver 512 using audio devices like Alexa, Siri and so on. The image-based assignment 512c may be communicated to parents and caregivers 512 either on the platform where the profile of the patient exists or directly to the device of the parents and caregiver 512 using messaging Apps like WhatsApp, Telegram and so on. The video-based assignment 512d may be communicated to parents and caregivers 512 either on the platform where the profile of the patient exists or directly to the device of the parents and caregiver 512 using messaging Apps like WhatsApp, Telegram, and so on. The other type of assignment 512e may include gaming exercises and so on which may be communicated to parents and caregivers 512 either on the platform where the profile of the patient exists or directly to the device of the parents and caregiver 512 using the link of the gaming console or the App using which the game is to be played. The assignment recommendations may further include sensory toy details along with the guide using which the patient can access the toy, listening programs like bone conduction headphones which are can be by the patient to follow the instructions and perform the assignment as guided. In some cases, the assignment may include web links to some music albums or game links, and so on. It should be noted that above assignments are included as way of example, and the assignment recommendations may be in any format and type which allows the patient to perform certain home-based exercises that can continue the care between multiple therapy sessions.

Some of the examples of different kind of assignment includes: a text-based assignment 512a may be in the form of ‘Repeat /er/ sound 10 times’ and so on. An audio-based assignment 512b, an image-based assignment 512c and a video-based assignment 512d may include an exercise based on the shared audio, video or image, respectively.

The communication module 510 may communicate either text-based assignment 512a, audio-based assignment 512b, image-based assignment 512c, video-based assignment 512d, others 512e alone or a combination thereof. This provides a wide range of assignment that will motivate the child to complete the tasks before going to the next therapy session. This will help in maintaining the overall continuity of treatment of the patient.

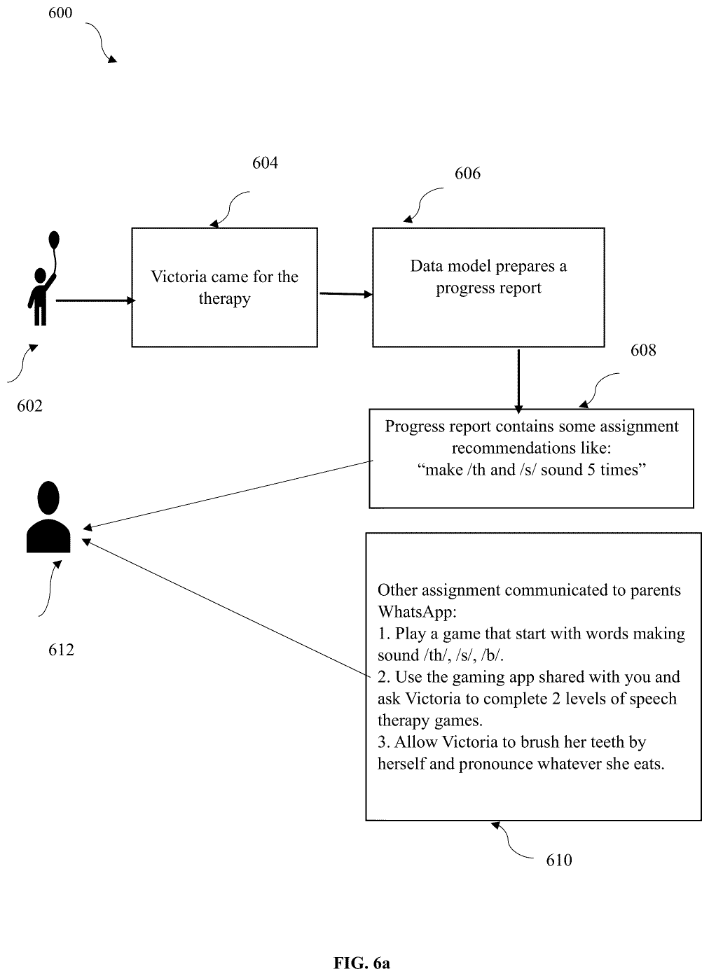

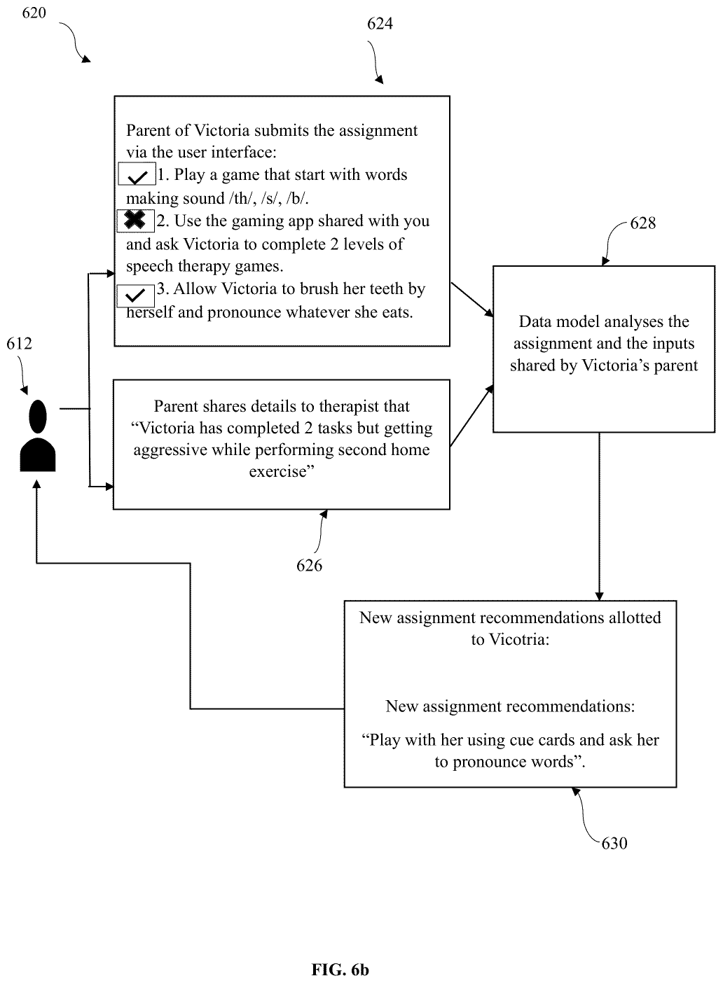

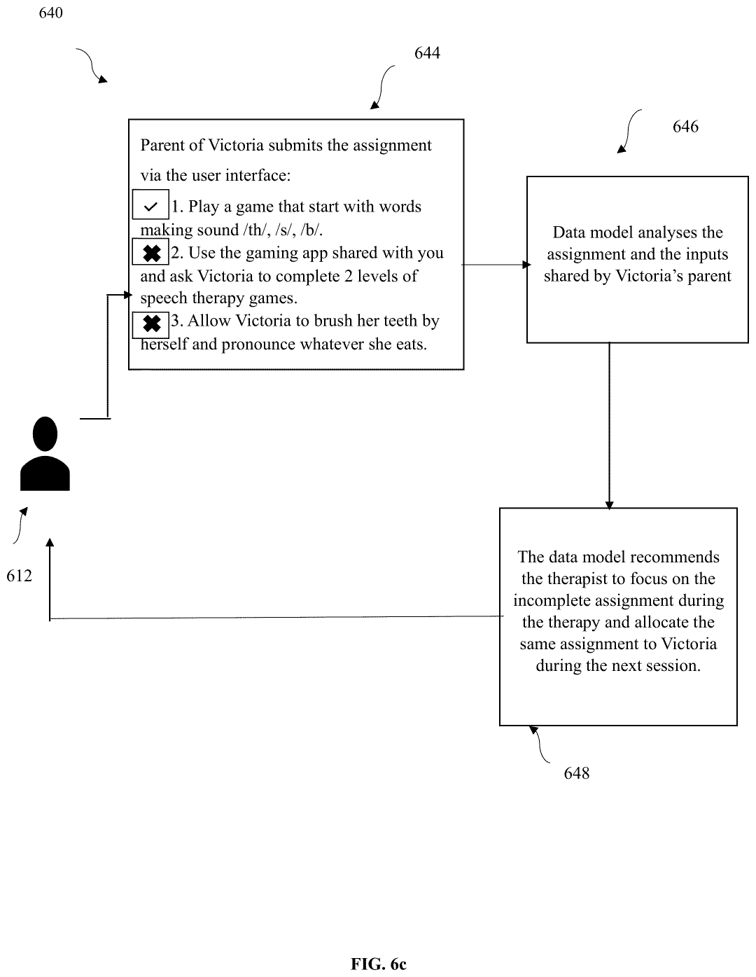

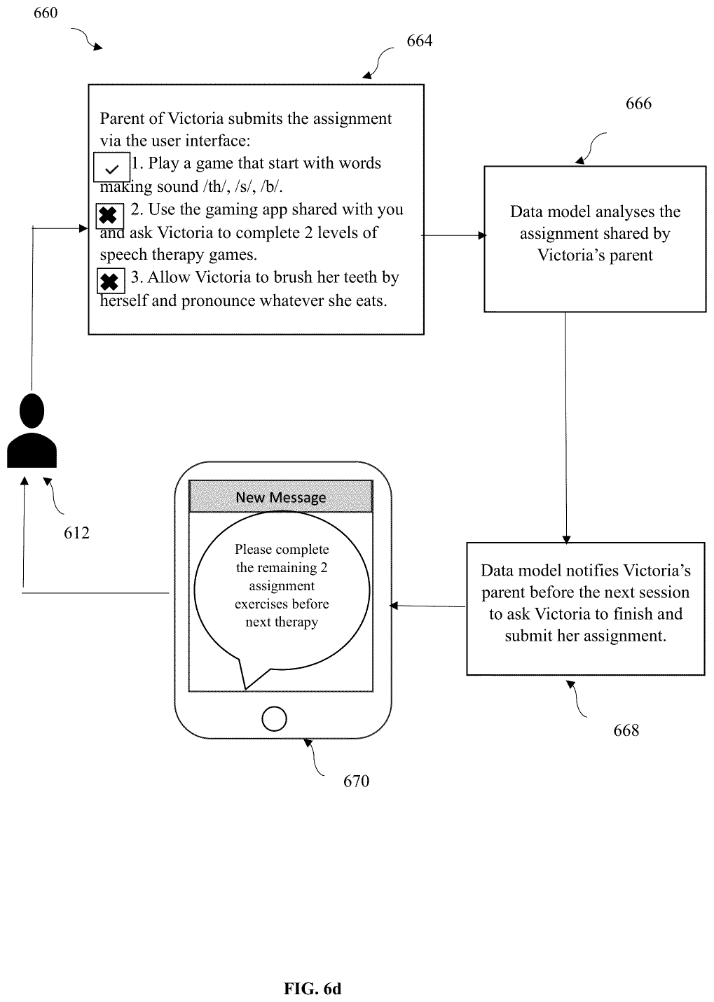

FIG. 6a illustrates an exemplary method 600 of generating an assignment recommendation for a patient undergoing therapy, in accordance with one implementation of the present disclosure.

As shown, the child named Victoria 602 comes to attend her therapy 604 with her parent 612. The expert who is doing her treatment i.e., her therapist accesses the medical data of the Victoria 602 using a user interface (not shown in the FIG. 6a). Based on the accessed data and the session notes, a progress report 606 is automatically generated based on her therapy session. As discussed earlier, the progress report 606 is generated by the data model and include SOAP notes of the therapy session. The progress report includes detailed version of Subjective, Objective, Assessment, Planning performed during the therapy session. It also includes one or more assignment recommendations 608. As shown, the assignment recommendations presented in the progress report 608 include “Make /th/ and /s/ sound 5 times”. This automatically generated progress report using data model 606 along with one or more approved assignment recommendation 608 can be accessed by the parent 612 of Victoria 602.

Further a detailed approved assignment recommendation 610 is communicated to the parent 612 on his device. The device may include smartphone, tablet, computer, laptop and so on. The one or more approved assignment recommendations 610 is communicated to the parent 612 in one or more formats which may include text, image, audio, video, gaming console and so on. As shown, the one or more assignment recommendations 610 here includes:
“1. Play a game that start with words making sound /th/, /s/, /b/.
2. Use the gaming app shared with you and ask Victoria to complete 2 levels of speech therapy games.
3. Allow Victoria to brush her teeth by herself and pronounce whatever she eats.”

FIG. 6b illustrates an exemplary method 620 which discloses an interaction between the parent and an expert when the patient faces problems while completing the assignment, in accordance with one implementation of the present disclosure.

As shown, the parent 612 of Victoria has received the one or more approved assignment recommendations on his device via WhatsApp (shown as an example, in this case). It should be notes that the assignment recommendation can be received on any other suitable App like Facebook messenger, Skype, Apple's FaceTime, text messages on handheld device, Alexa, Siri and so on. Also, the parent 612 can directly access the one or more approved assignment recommendations directly through the platform. In this example, it is shown that the parent 612 has received the one or more approved assignment recommendations via WhatsApp and will interact with the expert related to his/her queries via the WhatsApp user interface.

The parent 612 submits the one or more approved assignment allotted to Victoria 624 indicating a ‘tick mark against the completed task’ and ‘cross mark against the incomplete task’. The parent 612 submits the status of assignment 624, where the first and third tasks are completed and the second task is incomplete:
“1. Play a game that start with words making sound /th/, /s/, /b/.
2. Use the gaming app shared with you and ask Victoria to complete 2 levels of speech therapy games.
3. Allow Victoria to brush her teeth by herself and pronounce whatever she eats.” Further, the parent 612 submits using user interface an input to the expert 626 stating that “Victoria has completed 2 tasks but getting aggressive while performing the second home exercise”.

As soon as the data model receives the assignment status update 624 and input 626 by the parent 612, it starts analyzing 628 the one or more approved assignment recommendations 624 submitted by the parent 612. It also analyzes the input 626 shared by the parent 612 and processes both the data and using communicates feedback to the parent 612. The data model allots new assignment recommendations 630 to Victoria and communicate the same to the parent 612. The new assignment recommendations 630 allotted to Victoria includes: “Play with her using cue cards and ask her to pronounce words”. The data model immediately suggest the parent 612 to stop the second exercise as the child is getting aggressive because of that and this exercise is creating a negative impact towards the treatment of the child.

Hence, it can be seen that the data model provides personalized care plan to each patient based upon real time updates received by the patient and/or care giver of the patient.

FIG. 6c illustrates an exemplary method 640 which discloses an interaction between the parent and an expert when the patient does not complete the entire assignment, in accordance with one implementation of the present disclosure.

As shown, the parent 612 of Victoria has received the one or more approved assignment recommendations on his device via WhatsApp (shown as an example, in this case). In this example, it is shown that the parent 612 has received the one or more approved assignment recommendations via WhatsApp and will interact with the expert related to his/her queries using this user interface, however, the parent 612 can access the recommendations via the platform (also referred as Ocean Friend's Dashboard).

The parent 612 submits the one or more approved assignment allotted to Victoria 644 indicating a ‘tick mark against the completed task’ and ‘cross mark against the incomplete task’. As shown, the parent 612 submits the status of assignment 644 indicating the first task is completed (marked with a tick) while the other two are incomplete (marked with a cross) that:
“1. Play a game that start with words making sound /th/, /s/, /b/.
2. Use the gaming app shared with you and ask Victoria to complete 2 levels of speech therapy games.
3. Allow Victoria to brush her teeth by herself and pronounce whatever she eats.”

As soon as the data model receives the assignment status update 644 by the parent 612, it starts analyzing 646 the one or more approved assignment recommendations 644 submitted by the parent 612. The data model 648 “recommends the therapist to focus on the incomplete assignment during the therapy and allocates the same assignment to the Victoria during next session”. This means that the data model will recommend the therapist to focus on the part of the one or more approved assignment recommendations that were not completed by the child that was allotted to her to be completed in-between the two sessions i.e., before the start of the next session. Since she has not completed her allotted task so she might be missing the continuity of the treatment, hence the therapist is asked to focus on that specific area during the therapy. Further, after the therapy the one or more approved assignment recommendations allotted to Victoria and communicated to her parents 612 will include the same tasks that were left incomplete by her previously along with some new tasks.

In this way, the current system ensures that the patients do not miss on any assignments or home exercises allotted to them and the continuity of the treatment is maintained.

Further, FIG. 6d illustrates another exemplary method 660 which discloses an interaction between the parent and an expert when the patient does not complete the entire assignment and the notification is shared with the parent before the upcoming session, in accordance with one implementation of the present disclosure.

As shown, the parent 612 of Victoria has received the approved assignment recommendations on her device via WhatsApp (shown as an example, in this case).

The parent 612 submits the one or more approved assignment allotted to Victoria 664 indicating a ‘tick mark against the completed task’ and ‘cross mark against the incomplete task’. The parent 612 submits the status of assignment 664 that the first task is completed while the second and third tasks are incomplete:
“1. Play a game that start with words making sound /th/, /s/, /b/.
2. Use the gaming app shared with you and ask Victoria to complete 2 levels of speech therapy games.
3. Allow Victoria to brush her teeth by herself and pronounce whatever she eats.”

As soon as the data model receives the assignment status update 664, the data model analyzes 666 the assignment shared by the parent 612. The data model sends a notification 668 to the parent 612 stating that “Please complete the remaining two exercises before the next therapy” 670. The parents 612 are continuously reminded by sending the notifications on their device till the time they submit all the assignment recommendations allotted to the child. This helps in maintaining a seamless communication between the expert and the parent 612 and hence enhances the continuity of the treatment of the child.

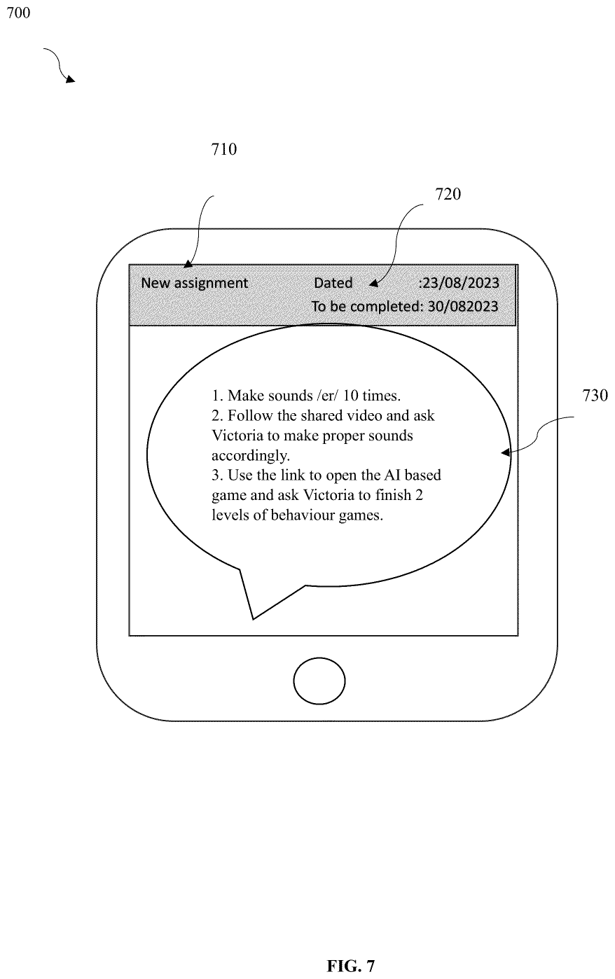

FIG. 7 illustrates an example implementation which discloses how a notification of new assignment recommendations 710 is communicated on parents and/or caregiver device 700, in accordance with one implementation of the present disclosure.

The exemplary view of the parents and caregiver device 700 disclosed herein is a mobile device 700 which is used by the parents of the patient to receive the notifications, one or more approved assignment recommendations 730 and so on. However, the device 700 used by the parent may also include laptop, computer, tablet or any similar kind of device. Further, the one or more approved assignment recommendations 730 may also be communicated to person other than parents of the patient which may include caregivers, guardian, another physician and/or therapist and so on.

The parents and caregiver device 700 include a notification at the top right disclosing “New Assignment” 710 and the current date and the due date 720 on or before which the one or more approved assignment recommendations 730 needs to be completed by the patient and submitted to the platform by the parent and caregiver of the patient. The parent and caregiver may directly contact the expert in case they have any queries related to the one or more approved assignment or any other queries related to patient's development and so on. The parents and caregiver device 700 receives the one or more approved assignment recommendations 730 that are automatically generated by the data model and communicated to the remote user which may include parents, caregivers, guardians of the patient. In the following example, the one or more approved assignment recommendations 730 includes:
“1. Make sounds /er/ 10 times.
2. Follow the shared video and ask Victoria to make proper sounds accordingly.
3. Use the link to open the AI based game and ask Victoria to finish 2 levels of behavior games.”

The parent and caregiver may directly access the one or more approved assignment recommendation 730 directly using the mobile messaging App, like in the case of this example.

Furthermore, the parents and caregiver device 700 may receive the notification directly on the communication Apps like WhatsApp, telegram, SMS and so on from which they have registered onto the platform. Also, the same one or more approved assignment recommendations is provided on the platform. This provides an ease of access to the parents and caregivers that they do not have to open the platform again and again to communicate with the expert, review the progress of the child, access the one or more assignment recommendations and so on. The parent and caregiver can directly get the notification on their mobile phone, tablet, computer or any other device linked with the platform. The notification may include alert for completion of one or more approved assignment recommendations, one or more assignment recommendations in-between the therapy sessions and so on.

Also, the parent and caregiver may control the level of assignment that are assigned to the patient based on the initial input i.e., during the start of the therapy or treatment the parents are asked to fill in the details like how many hours they can work with the patient on completing the assignment. In a different scenario, the parent and caregiver may also edit these settings in real time using the platform. In an exemplary scenario, if a parent enters that they can allot 10 hours/week for the patient's assignment, then the one or more approved assignment recommendations will be allotted to them on that basis. If suppose a parent enters that they cannot allot any time for patient's assignment then the patient will not be provided any assignment-based task. The data model will allot simple tasks which do not consume time like the music will play when child enters home, all the day to day activities of the patient needs to be monitored and to be completed by the patient in an orderly manner and so on. In short, the one or more approved assignment recommendations are provided to the patient dynamically based on the engagement of the parent and caregiver of the patient.

As described throughout this application the term “assignment recommendations” is used to mean one or more home exercises allocated to the patients in-between the therapy to maintain the continuity of care using system, application, or any other similar software platform. Also, the terms “parents”, “caregivers”, “users” or “members” will have the similar meaning throughout the disclosure. In addition, the system of the present disclosure is relatively inexpensive, safe and easy to use.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

### Technical Advancements

The present disclosure described herein above for enhancing the continuity of care for a patient has several technical advantages including, but not limited to, the realization of:
automatically generates assignment recommendations without manual intervention;
reduces time of the physician/therapist in documentation;
suitable for predicting the progress of health condition;
user friendly;
easy to use;
versatility of the user interface;
provides seamless communication between parents and expert;
enhances continuity of care in a patient's treatment;
helps the patient and expert during the treatment;
increases bond between parents and patient;
maintains transparency and security of the data.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

We claim:

1. A method to enhance the continuity of care for a patient comprising:
accessing the patient's data available in one or more formats, wherein the patient's data includes pre-stored data, real-time generated data, or a combination thereof;
processing the patient's data using machine learning techniques and a language learning model by extracting relevant information from the patient's data;
generating one or more patient-specific assignment recommendations automatically in correspondence with the processed patient's data, wherein the one or more assignment recommendations are designed based on a correlation between the pre-stored data and the real-time generated data in order to address a condition management in the patient;
receiving one or more input from an expert on one or more assignment recommendations to generate one or more approved assignment recommendations;
communicating the one or more approved assignment recommendations to one or more remote users including, parents and caregivers of the patient, wherein the parents and caregivers may access and review the approved one or more assignment recommendations.

2. The method as claimed in claim 1, wherein the patient's data comprises medical session report, progress report, patient's health records, or a combination thereof, where the medical session report further comprises one or more previous or ongoing session reports, the progress report comprises one or more medical session reports clubbed to define progress made by the patient over a period, and the patient's health record comprises one or more additional health related details of the patient.

3. The method as claimed in claim 2, wherein the medical session report and the progress report comprises one or more SOAP notes such that each SOAP note further comprises details related to subjective information, objective observations, assessment findings, plans for future treatment of the patient and optionally includes one or more assignment recommendations.

4. The method as claimed in claim 1, wherein the pre-stored data comprises one or more medical session reports or progress reports of the patient including SOAP notes of the previous sessions indicating assessment of the condition and progress made by the patient in previous sessions.

5. The method as claimed in claim 1, wherein the real-time generated data comprises session notes of the ongoing session, patient's data, medical data of one or more patients with similar condition or a combination thereof.

6. The method as claimed in claim 1, wherein the patient's data may be in one or more of the following formats-text, audio, video, image, recording of the session or a combination thereof.

7. The method as claimed in claim 1, wherein the one or more approved assignment recommendations may be communicated to the one or more remote users in one or more of the following formats-text, audio, video, image, gaming task, recording, or a combination thereof.

8. The method as claimed in claim 1, wherein the one or more assignment recommendations are generated based on the session notes of the ongoing and previous sessions, medical data of one or more patients with similar condition, where the assignment recommendations are tailored according to the patient's requirement and generated to improve patient's condition.

9. The method as claimed in claim 1, wherein the one or more remote user may share an update with the expert related to the communicated one or more approved assignment recommendations, thereby facilitating a seamless communication between the one or more remote user and the expert in order to enhance the continuity of care for the patient.

10. The method as claimed in claim 1 establishes a user-query interface to enable one or more remote users to pose questions or seek clarification from the expert regarding the one or more approved assignment recommendations and/or one or more health condition of the patient, wherein the expert may be a physician, therapist, clinician, doctor and/or any other person having experience in providing recommendations and/or treatment related to the condition of the patient.

11. The method as claimed in claim 1, wherein the one or more assignment recommendations is automatically generated by using machine learning model, which is then edited and/or modified by the expert based upon his assessment of the patient's condition or one or more preferences of receiving the assignment by the patient or caregiver to get the one or more approved assignment recommendations.

12. The method as claimed in claim 1, wherein the expert addresses user queries, provide guidance, and maintain an interactive consultation with the one or more remote user in order to maintain the continuity of care.

13. A system to enhance the continuity of care for a patient comprising:
a server including a memory to store instructions;
a processing device operatively coupled to the server and is configured to execute instructions to:
access the patient's data available in one or more formats, wherein the patient's data includes a pre-stored data, a real-time generated data or a combination thereof;
process the patient's data using machine learning techniques and language learning model based on extraction of relevant information from the patient's data;
generate one or more patient specific assignment recommendations automatically in correspondence with the processed patient's data, wherein the one or more assignment recommendations are designed based on a correlation between the pre-stored data and real-time generated data in order to address a condition management in the patient;
receive one or more inputs from an expert on one or more assignment recommendations to generate one or more approved assignment recommendations;
communicate the approved one or more assignment recommendations to one or more remote users which may include parents and caregivers of the patient, wherein the parents and caregivers can access and review the approved one or more assignment recommendations.

14. The system as claimed in claim 13, further comprises a data model configured to perform a contextual analysis of the pre-stored data and the real-time stored data in order to identify the intend behind the patient's data.

15. The system as claimed in claim 13, wherein the data model may be a machine learning models configured to process and analyse the patient's data using techniques such as convolutional neural networks, audio signal processing, or video signal processing.

16. The system as claimed in claim 13, wherein the data model further comprises a Generative AI model including one or more of GPT-3 (Generative Pre-trained Transformer 3), GPT-3.5, BERT (Bidirectional Encoder Representations from Transformers), ROBERTa (Robustly Optimized BERT Approach), ALBERT (A Lite BERT), T5 (Text-to-Text Transfer Transformer) or a combination thereof.

17. The system as claimed in claim 13, wherein the system further allows an interactive feedback loop between the expert and the one or more remote users which may include parents and/or caregivers of the patient in order to improve patient's engagement and treatment outcome.

18. The system as claimed in claim 13, wherein the expert accesses the pre-stored data and the real-time data in one or more formats via a user interface including a graphical user interface, a voice user interface, a video user interface, a gesture user interface, or combination therefor.

19. The system as claimed in claim 13, wherein the data model updates the one or more approved assignment recommendations and provides a dynamic response based on inputs provided by the one or more remote users.

20. The system as claim in claim 13 is configured to share notifications with the one or more remote users for completing and updating the one or more approved assignment recommendations.
